(19) United States
(12) Patent Application Publication
(10) Pub. No.: US 2005/0233976 A1
(43) Pub. Date: Oct. 20, 2005

(54) Therapeutic methods

(75) Inventor: Rodney Pearlman, El Granada, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: SAEGIA PHARMACEUTICALS, INC., HALF MOON BAY, CA (US)

(21) Appl. No.: 10/515,981

(22) PCT Filed: May 29, 2003
(86) PCT No.: PCT/US03/17161

§ 371 Date: Jun. 15, 2005

Related U.S. Application Data
(60) Provisional application No. 60/384,754, filed on May 29, 2002.

(51) Int. Cl. A61K 38/04; C07K 5/04

(52) U.S. Cl. 514/19; 548/537

(57) ABSTRACT

The invention provides methods for treating a symptom of MCI as well as methods for slowing the progression from MCI to AD.

### BACKGROUND OF THE INVENTION

Researchers in the field of aging and dementia are focusing their attention on identifying and characterizing the earliest stages of cognitive impairment. By identifying these stages, it may be possible to prevent, delay, or reverse disease-associated brain damage. For example, recent research has identified a condition known as mild cognitive impairment (MCI) that is distinct from the cognitive impairment associated with normal aging and with Alzheimer's disease (AD). See Friedrich, JAMA 282, 621-622 (1999); and Peterson et al., Arch. Neurol., 58, 1985-1992 (2001).

MCI is a clinical condition in which a person experiences a slight impairment in cognitive function, typically memory, with otherwise normal performance, yet does not meet currently accepted criteria for a diagnosis of AD. The symptoms of MCI include self-reported memory complaints, impaired memory function, preserved general cognitive function, intact activities of daily living, and a lack of dementia. Patients can be diagnosed on the basis of neuropsychological assessment, functional and structural neuroimaging, neuropatholgy, and the measurement of biomarkers, for example, different levels of neurochemicals in the cerebrospinal fluid.

Subjects with MCI develop AD at a rate of about 10% to 15% per year, whereas healthy control subjects develop AD at a rate of about 1% to 2% per year. Data from the Mayo Alzheimer's Disease Research Center, which has been observing a group of these subjects for more than 10 years, has demonstrated a conversion from MCI to AD of up to 80% during approximately 6 years. Consequently, MCI has been recognized as a suitable condition for possible therapeutic intervention

There is currently a need for methods for treating the symptoms of MCI as well as a need for methods to slow or prevent the progression from MCI to AD.

### SUMMARY OF INVENTION

The present invention provides a method of treating a symptom of MCI comprising administering to a mammal in need of such therapy, an effective amount of a compound of formula (1) 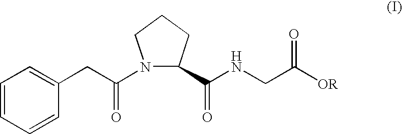
wherein R is (C₁-C₄)alkyl; or a pharmaceutically acceptable salt thereof.

The invention also provides the use of a compound of formula (1) 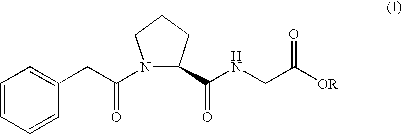
wherein R is (C₁-C₄)alkyl; or a pharmaceutically acceptable salt thereof, for the manufacture of a medicament useful for the treatment of a symptom of MCI in a mammal.

### DETAILED DESCRIPTION OF THE INVENTION

I. Definitions

“Mild cognitive impairment (MCI)” is a term that is understood in the art. MCI refers to a condition between the cognition of normal aging and mild dementia, in which a person experiences a slight impairment in cognitive function, typically memory, with otherwise normal performance, yet does not meet accepted criteria for a diagnosis of AD.

A “symptom of MCI” can be, for example, at least one cognitive change, e.g., an impairment of memory function compared to one's age cohort. For example, a “symptom of MCI” can be memory impairment that is at least one standard deviation below that of a subject's peers, but without significant impairment in activities of daily living. In addition, symptoms of MCI include, but are not limited to, reduced medial lobe volume, e.g., medial temporal lobe atrophy and/or reduced hippocampal volume; delayed recall tasks or verbal and nonverbal stimuli; a decline in linguistic function or verbal fluency, e.g., as measured by the Boston Naming Test. Additionally, a “symptom of MCI” can also be an impairment in a single cognitive domain other than memory, e.g., a pronounced language disturbance, which can progress to primary progressive aphasia, or an alteration in attentional abilities or comportment and a dysexecutive syndrome, which may progress to frontotemporal dementia As used herein, the term, “symptom of MCI” also includes the increased propensity for a patient to develop AD.

The term “reducing the progression from MCI to AD” includes reducing the deterioration of cognitive function by a measurable and statistically significant percentage in one or more of the following six standard tests: 1) Wechsler Adult Intelligence Scale—Revised (WAIS Digit Span—repeating string of digits forward and reverse; WAIS Digit Symbol—coded symbols from key), 2) Wechsler Memory Scale (Paired Associates Learning (PAL)—word pairs; Logical Memory (LM)—stories), 3) Brief Cognitive Rating Scale (BCRS) (Multi-axis assessment), 4) Randt Memory Test (Short story details 30 minutes after hearing), 5) Benton Revised Visual Retention Test (Geometric patterns to be remembered), and 6) Mini-Mental Status Exam (MMSE). Preferably, the deterioration of cognitive function is reduced by at least about 10 percent in one or more of these six standard tests; more preferably, the deterioration of cognitive function is reduced by at least about 20 percent in one or more of these six standard tests; and most preferably, the deterioration of cognitive function is reduced by at least about 50 percent in one or more of these six standard tests.

A preferred compound of Formula (I) useful in the methods of the invention is the compound C₆H₅CH₂—C(═O)-L-Pro-Gly-OC₂H₅ or a pharmaceutically acceptable salt thereof.

The ability of a compound to lessen one or more symptom of MCI can be determined using screening methods such as psychological tests and clinical assessment. Various cognitive screening tests and psychological tests, e.g., neuropsychological tests, are available, such as those employed in the following studies: Galuuzzi et al., Aging (Milano), 13, 183-202 (2001); Petersen et al., Arch. Neurol., 58, 1985-1992 (2001); Folstein et al., J. Psychiatr. Res. 12, 189-198 (1975); and Rosen et al., J. Psychiatr. 141, 1356-1364 (1984). For example, clinical assessment of the ability of a compound to treat a symptom of MCI can be made using one or more of the following tests:
1. Standardized set of methods, including the clinico-psychological, widely used neuropsychological, psychophysiological, electrophysiological and biochemical methods;
2. Clinical description of the effects of the compound of Formula I test dose based on clinical integrated expert evaluation of the main effect of the drug and determination, in each patient, of individual drug effects with estimation of the following types of pharmacological action: activating, tranquilo-activating, tranquilizing, tranquilo-sedative, sedative and polymorphous with polar changeover of the response within activation-sedation vector (Neznamov et al., in: “Biological Basis of Individual Sensitivity to Psychotropic Drugs”, Graffhan Press, Ltd, Gaviraghi et al, ed., Edinbourgh (1994));
3. Use of the Unified Symptom Severity Scale for assessment of clinico-pharmacological action of psychotropic drugs in patients with mild psychological disorders enables obtaining quantitative data characterizing changes in psychopathologic symptomatology and profile of psychotropic action of drugs;
4. Brief Cognitive Rating Scale (BCRS) measures some cognitive components (the concentration of the attention, short-term and long-term memory, orientation, activity) and scores the cognitive deterioration (the total score higher than 5 represents the pronounced disorders (Reisberg et al., J. Psychopharm. Bull., 19, 47-50 (1983)));
5. Mini Mental State Evaluation (MMSE) consists of a battery of neuropsychological tests assessing arbitrary scores of cognitive functions (attention, memory, gnosis, speech, praxis, count) (the higher score represents the better state of cognitive function. (Folstein et al., J. Psychiatr. Research, 12, 189-198 (1975)));
6. Cognitive Capacity Screening Exam (CCSE) consists of a battery of tests estimating cognitive functions (orientation, memory, count, ability to make conclusions, ability to classify subjects) (the higher score represents the better state of cognitive function (Jacobs et al., Annals of Intern. Medicine, 86, 40-46 (1977)));
7. Spielberger-Khanin situational anxiety test is oriented on arbitrary evaluation of reactive (situational) anxiety experienced by the patient (Hanin, “Short guide to the usage of Spielberg' scale of personal anxiety,” 5-21, Leningrad (Rus.) (1976));
8. Self-Assessment of Functional State (SAFN) can be used for a subjective evaluation of drug effects on self-feeling, activity and mood (Doskin et al., Vopr. Psychologii. N6, 141-145 (1973));
9. The Overall Clinical Impression Scale (OCIS) is used for quantitative estimation of therapeutic efficacy of the drug, its endurance and safety on the basis of such variables as severity of illness, general improvement, therapeutic effect, and side-effects (National Institute of Mental Health: 12-CGI. Clinical global Impression; ECDEU Assessment Manual for Psychopharmacology, Guyo ed., Rockville, Md., 217-222 (1976)); and
10 The following computerized method for assessment of psychophysiological state can be conducted: the procedure estimates the following parameters: 1) Time of simple motor reaction to light signal (sec); 2) Allocation of attention (sec); 3) Volume of attention (sec); 4) Stability of attention (sec); 5) Choice of light signals (sec); 6) Amount of choice errors (in %); 7) Volume of short-term visual memory (arbitrary units); 8) Response to moving object (arbitrary units); 9) Percent of hits in the moving object; 10) Integral measure of performance in the test (arbitrary units) (Morozov et al., Experiment, and Clinic. Pharmacol. 55, 68-70 (1992); Morozov et al. “Complex Computerized Methods for the Evaluation of the Psycho-Physiological State and Operatory Worling Ability,” In: Functional State of the Man and Methods of its Analysis. 3-8, Moscow (Rus.) (1992)).

The compounds can be formulated as pharmaceutical compositions and administered to a mammalian host, such as a human patient in a variety of forms adapted to the chosen route of administration, e.g., orally, parenterally, by intravenous, intramuscular, or subcutaneous routes, transdermal (passive or iontophoretic) or via suppository.

Thus, compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The compounds may also be administered intravenously or intraperitoneally by infusion or injection. Solutions of the agent or its salt can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the compound in the required amount in the appropriate solvent with various of the other ingredients enumerated above. In the case of sterile powders for the preparation of sterile injectable solutions, the preferred methods of preparation are vacuum drying and freeze drying techniques, which yield a powder of the active ingredient plus any additional desired ingredients present in the sterile solutions.

Useful dosages of the compounds can be determined by comparing their in vitro activity, and in vivo activity in animal models. Methods for the extrapolation of effective dosages in mice, and other animals, to humans are known to the art; for example, see U.S. Pat. No. 4,938,949. However, the amount of the compound, or an active salt or derivative thereof required for use in treatment will vary not only with the particular agent selected but also with the route of administration, the nature of the condition being treated and the age and condition of the patient and will be ultimately at the discretion of the attendant physician or clinician.

In general, however, a suitable dose will be in the range of from about 0.5 to about 100 mg, e.g., from about 1.0 to about 75 mg per day, preferably in the range of 6 to 90 mg/day, and most preferably in the range of 15 to 60 mg/day.

The compound is conveniently administered in unit dosage form; for example, containing 0.25 to 100 mg, conveniently 0.5 to 75 mg, most conveniently, 5 to 30 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The invention will now be illustrated by the following non-limiting example.

### EXAMPLE 1

To determine if a compound of Formula I is effective for treating a symptom of MCI a Phase I clinical study in normal volunteers can be conducted. Cognitive enhancement properties, such as improved attention span and stability, improved fine motor coordination, improved long-term memory of auditory signals and improved spatial and logical thinking indices of a compound of Formula I can be compared with a placebo. For example, effects can be observed after a single dose of 20 mg of a compound of Formula I as well as following 20 mg daily for a 13-day course. Phase II clinical trials in patients with cognitive disorders can be conducted. For example, a dose of 15-20 mg/day can be administered to determine effectiveness and tolerance. Exemplary trials are summarized in Table 1.

TABLE 1

| Study | n | Dose/Duration | Population Description |
|---|---|---|---|
| 1 | 20 | 20 mg test dose, then 20 mg/day for | Normal Volunteers |
| | | 13 d | |
| 2 | 20 | 10 mg test dose, then 30 mg/day for | Mild Cognitive |
| | | 28 d | Disturbance |
| 3 | 20 | 5 mg test dose, then 15 mg/day for | Mild Cognitive |
| | | 28 d | Disturbance |
| 4 | 20 | 20 mg, single dose | Normal Volunteers - |
| | | | Pharmacokinetics |

Clinical Pharmacology

By virtue of their activity as cognitive enhancing agents, compounds of Formula I affect central nervous function in a variety of ways and in the absence of significant sedation and/or excitability. The ability of compounds of Formula I to treat a symptom of MCI can be carefully evaluated during one Phase I and two Phase II studies with the aim of selecting a dose regimen that provides therapeutic effects on cognitive function with minimal adverse effects. Effects on the following parameters can also be measured: spatial and logical thinking, fine motor coordination, short and long-term memory, counting and logistical task performance. For example, the effects of compound I can be assessed in a group of patients with cognitive disturbances, the outcome of which, for example, could support the selection of a dose of 30 mg daily of compound I.

Phase I safety, tolerability, and pharmacokinetic properties of a compound of Formula I can be determined as follows:

Efficacy, Tolerability and Safety of a Compound I of Formula I Compared to Placebo.

Methods

Methods

A single oral dose of 20 mg of a compound of Formula I or placebo is administered daily to 20 healthy male volunteers for 13 consecutive days. Operational task performance is evaluated using computer programs that include a series of tests that are known to be useful to measure sensory and motor function. Psychological testing include MMPI questionnaires, the Spielgerger-Khanin situational anxiety test and a Self-Assessment of Functional State instrument. Memory can be evaluated with multiple standardized tests of short- and long-term recall and attention was evaluated according to several standardized performance tests. Speed and logic operations are estimated using standard counting, subtraction and quantitative reasoning relational tests. Multiple psycho-physiological assessments can be made at pre-determined intervals, including: “reaction to the moving object”; “simple reaction time to light and sound stimulation”; “attention (span, stability and distribution)”; “short and long-term visual memory”; “critical frequency of flashing light fusion”; “ability to choose between two visual or three auditory signals”; “ability to reproduce motor reactions to various operations”; “spatial and logical thought assessments”. All tests can be administered at the beginning and at the end of each study day following a compound of Formula I or placebo dose administration.

Study of Tolerance, Safety and Characteristics of the Psychotropic Action of a Compound of Formula I in Patients with Mild Cognitive Disturbance

Methods

At study entry, patients fit pre-defined criteria for MCI.

10 mg of a compound of Formula I is given orally to 20 patients with MCI on Study Day 1 followed by 10 mg t.i.d. orally for 28 days. By protocol, if fewer than 2 of the first 4 patients do not demonstrate efficacy during the 28 days of t.i.d. treatment the test dose can be increased to 20 mg and the repeated dose increased to 20 mg t.i.d.

Analysis of clinical manifestations of its action (the Self Assessment of Functional State, the Spielberger-Khanin test, a psycho-physiological test battery and an EEG examination) is administered at baseline and 1, 3 and 24 hours after administration of the test dose. Effect on nocturnal sleep is assessed after 24 hours.

Upon initiation of ongoing daily therapy, following successful administration of the test dose, further assessments can be made, for example, on study days 7, 14, 21 and 28. Assessments can include those taken following the test and, in addition, a Uniform Symptom Severity Score, Brief Cognitive Rating Scale, Mini Mental Status Exam, Cognitive Screening Exam and Overall Clinical Impression Scale (OCIS).

Study of Tolerance, Safety and Efficacy of a Compound of Formula I in Patients with Mild Cognitive Disturbance

Methods

At study entry, patients fulfilled pre-defined criteria for MCI.

5 mg of a compound of Formula I is given orally on Study Day 1 to 14 patients with MCI, followed by S mg t.i.d. orally for 28 days.

Analysis of clinical manifestations of its action, e.g., improvement in their overall clinical condition, is administered at baseline, immediately after the test dose and at scheduled intervals following administration of the test dose.

Pharmacokinetics and Metabolism in Humans

The pharmacokinetics of a compound of Formula I can be investigated in healthy volunteers. For example, a compound of Formula I can be investigated in healthy volunteers after a single oral dose of 10 mg.

Concentrations of a compound of Formula I can be determined in plasma by validated high-performance liquid chromatography (HPLC) methods with ultraviolet detection

Methods

Single oral doses of 20 mg a compound of Formula I (two tablets of 10 mg each) can administered to healthy volunteers, for example, following a light breakfast. Concentrations of a compound of Formula I can be determined in plasma as described above, and the maximum plasma concentrations (Cₘₐₓ) and plasma tₘₐₓ as well as and adverse events can be determined.

Metabolism

Metabolism of a compound of Formula I can be investigated in healthy volunteers who, for example, can be administered 20 mg of an unlabelled compound of Formula I. Following administration, elimination of the compound from the plasma (tₘₐₓ) can be determined.

The identification of metabolites of a compound of Formula I in humans can be investigated.

Efficacy in Phase II Studies

Methods

At study entry, patients should fulfill pre-defined criteria for MCI.

In the evaluation of efficacy data from a Phase II study, the Overall Clinical impression Scale, specifically, the General improvement Index, can be used to determine whether or not a patient is a “responder” to a compound of Formula I (as defined per protocol), which can be used as the primary efficacy outcome measure. The definition of responder, for example, can require that a patient have a score of ≦2 points on both the General Improvement Index and the Efficacy Index.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

2. The method of claim 1 wherein the symptom of MCI is an impairment of memory function compared to one's age cohort; memory impairment that is at least one standard deviation below that of a subject's peers, but without significant impairment in activities of daily living; reduced medial lobe volume; delayed recall tasks or verbal and nonverbal stimuli; a decline in linguistic function or verbal fluency; or impairment in a single cognitive domain other than memory.

3. The method of claim 1 wherein the symptom of MCI is the progression from MCI to AD.

4. The method of claim 1 wherein the symptom of MCI is a symptom other than the progression from MCI to AD.

5. The method of claim 3 wherein the rate of progression from MCI to AD is reduced by at least about 10 percent.

6. The method of claim 3 wherein the rate of progression from MCI to AD is reduced by at least about 20 percent.

7. The method of claim 3 wherein the rate of progression from MCI to AD is reduced by at least about 50 percent.

8. The method of claim 1 wherein the compound is C₆H₅CH₂—C(═O)-L-Pro-Gly-OC₂H₅, or a pharmaceutically acceptable salt thereof.

9-16. (canceled)
